(19) United States
(12) Patent Application Publication
(10) Pub. No.: US 2016/0051663 A1
(43) Pub. Date: Feb. 25, 2016

(54) METHODS AND DEVICES FOR CANINE HERPESVIRUS 1 (CHV-1) DETECTION AND PREVENTION

(71) Applicant: THE BOARD OF REGENTS FOR OKLAHOMA STATE UNIVERSITY, STILLWATER, OK (US)

(72) Inventor: Sanjay Kapil, Stillwater, OK (US)

(21) Appl. No.: 14/831,265

(22) Filed: Aug. 20, 2015

Related U.S. Application Data
(60) Provisional application No. 62/039,592, filed on Aug. 20, 2014.

(51) Int. Cl. A61K 39/245; A61K 9/00; A61K 31/522; A61F 6/06; A61K 31/7068; A61K 31/7076; C12Q 1/70; C12N 7/00; A61K 31/7072

(52) U.S. Cl. CPC A61K 39/245; C12N 7/00; A61K 9/0034; A61K 31/522; A61K 31/7072; A61K 31/7068; A61K 31/7076; C12Q 1/705; A61F 6/06; A61K 2039/5254

(57) ABSTRACT

Methods for detection of canine herpes virus 1 (CHV-1) in archived paraffin-embedded tissue are provided. The methods use CHV-1 glycoprotein B (gB)-specific PCR to detect the CHV-1. A vaginal sleeve that protects puppies from contracting CHV-1 by contact with the mother's vaginal wall during birth is also provided. The sleeve is inserted into the vagina of a pregnant female dog prior to birth and puppies traverse the birth canal through the sleeve without coming into direct contact with vaginal tissue. Compositions and methods of immunizing against CHV-1 in dogs of all ages are also provided.

### CROSS REFERENCE TO RELATED APPLICATIONS

This application claims the benefit of U.S. Provisional Patent Application Ser. No. 62/039,592 filed on Aug. 20, 2014, and incorporates said provisional application by reference into this document as if fully set out at this point.

### TECHNICAL FIELD

This disclosure generally relates to methods of detecting CHV-1, to devices and methods of preventing transmission of CHV-1, and to compositions and methods of immunizing animals against CHV-1. In particular, CHV-1 is detected in archived fixed, paraffin embedded samples; transmission of CHV-1 from mother to offspring during birth is prevented by the use of a vaginal sleeve, e.g. for litters of puppies; and compositions and methods of immunizing against CHV-1 in dogs of all ages are provided.

### BACKGROUND

Canine herpesvirus (CHV-1) is an alpha herpesvirus that is well documented to infect newborn puppies. The infection is transmitted by contact with secretions and excretions of carrier dogs housed in groups (1). CHV-1 is widespread in canine populations based on serum antibody prevalence in different countries (36% to as high as 94%) in the Western hemisphere (1). Canine herpesvirus also serves as an animal model for herpes simplex virus (HSV) in people, and similar strategies can be applied for both canine and human patient management.

The role of CHV in canine disease is still not completely understood because large scale population studies have not been conducted. This lack of CHV-1 information has prevented mass containment of CHV-1 infections worldwide. In fact, some information in the literature is incorrect because accurate methods for detection of CHV-1 are not available, and issues involved in CHV-1 detection for different scenarios are not completely understood. For example, diagnostic issues differ according to whether shedding of active CHV-1 is measured in an adult pregnant female dog, in secretions and/or in samples of epithelial surfaces, and in external secretions and cavities and/or samples of epithelial surfaces in a puppy, or any other stage of life of a dog. Further, critical invention points for management of CHV-1 have not been defined to control CHV-1 in populations. Transmission routes are incompletely defined and routes of transmission that help to perpetuate infection during the perinatal period have not been studied. Further, the role of latent CHV-1 infection and latency-associated mechanisms for CHV are unclear.

The bottle necks in epidemiology of CHV-1 infections have led to wide spread infection. However, due to a lack of appreciation of the extent of the clinical problems, the preventive tools are not commercially available in the United States and many other parts of the world (other than parts of the European Union). In a recent study in Germany, CHV was not detected in sick and healthy dogs with respiratory disease using the polymerase gene as a target for PCR (2). However, the selection of a suitable PCR target can make a difference in the sensitivity of the reaction, so this result may be inaccurate. CHV-1 is known to circulate in animal shelters due to exposure of naïve animals to dogs shedding CHV-1 as a result of close contact between animals kept in groups (3). Three forms of diseases due to CHV-1 have been documented: reproductive disease with abortions and stillbirths, respiratory distress and conjunctivitis. Other possible forms of CHV-1 disease such as acute encephalitis have not been documented, likely due to a lack of suitable diagnostic tools in veterinary diagnostic laboratories.

All CHV syndromes are caused by a virus that has potentially only one serotype, strains of which are closely related genetically worldwide (4). Thus, epidemiological studies cannot distinguish between continuous circulation of the same virus and/or frequent reintroduction of new CHV isolate. Due to differences and mutations in the DNA sequences of CHV-1, a vaccine based on a recent isolate of the most prevalent strain of CHV-1 in a geographic region would be preferred for vaccine preparation.

CHV-1 problems are widespread in dogs that are housed in groups. However, diagnostic methods available to veterinary diagnostic laboratories, such as histopathology for CHV-1 detection, lack sensitivity. As a result, health problems due to CHV-1 are frequently undiagnosed. Further, methods for performing retrospective work with CHV-1 using formalin-fixed, paraffin-embedded tissue blocks are not well described or validated. Canine herpesvirus is an enveloped virus and its survival in tissues is attenuated due to proteolysis and degradation during transport to diagnostic laboratories. Unfortunately, in the US there are currently no USDA restrictions on movement of canine germplasm and live dogs for breeding, so that indiscriminate importation from countries where CHV-1 infections are rampant is permissible.

Thus, what is needed are systems and methods for rapid, sensitive and specific detection of CHV-1 in antemortem specimens (e.g. vaginal epithelial and other specimens) and in archived specimens (e.g. paraffin-embedded tissue blocks such as those from kennels or other facilities with CHV-associated problems, whether newly discovered or ongoing). The provision of such systems and methods enables correct diagnostic evaluations and permits the identification and availability of recent American CHV-1 isolates for much needed vaccine production. Further, there is a need in the art to protect dogs, e.g. newborn puppies, from exposure to CHV-1 e.g. during birth when the mother is infected, or by contact with other dogs housed in group conditions.

Before proceeding to a description of the present invention, however, it should be noted and remembered that the description of the invention which follows, together with the accompanying drawings, should not be construed as limiting the invention to the examples (or embodiments) shown and described. This is so because those skilled in the art to which the invention pertains will be able to devise other forms of this invention within the ambit of the appended claims.

### SUMMARY OF THE INVENTION

Provided herein are systems and methods for rapid, sensitive and specific detection of CHV-1 in both ante- and postmortem paraffin embedded specimens from dogs suspected of being infected with the virus. Exemplary embodiments include a PCR based method that uses Canine Herpes Virus Glycoprotein B (CHVgB)-specific PCR as a test for CHV-1.

Diagnostic and management approaches to prevent transmission of CHV-1 in populations in kennels, e.g. to susceptible naïve newborn animals, are also provided. The approaches are based on information provided herein that highlights the need for systematic management of CHV-1 epizootics in group housed and breeding dogs. Similar principles may be applied to alpha herpesvirus infections in other carnivores and to herpes simplex virus infections in humans.

Also provided are vaccines and immunogenic compositions that, when administered to a subject such as a canine, elicit an immune response to CHV-1 in the subject, e.g. a protective immune response. Protocols for developing such immunogenic compositions and vaccines to prevent CHV-1 infections are provided, as are uses for the immunogenic compositions/vaccines to prevent or attenuate the spread of CHV-1 infection in susceptible individuals and in groups of susceptible individuals. Various other prevention and treatment methods are also described, including immunotherapy protocols using CHV-1 immune serum to prevent mortality in infected individuals.

According to another embodiment, there is provided a vaginal sleeve that is used in connection with female canines to prevent CHV-1 transmission to newborns during delivery through the birth canal.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1. Schematic diagram of a canine vaginal sleeve inserted into the vagina of a female dog.

FIG. 2 contains a design of an exemplary embodiment of a multi-puppy “purse-string” vaginal sleeve.

FIG. 3 contains a further embodiment of a canine vaginal sleeve.

FIG. 4 contains a further embodiment of a canine vaginal sleeve.

FIG. 5 contains a further embodiment of a canine vaginal sleeve.

FIG. 6 contains an alternate view of the canine vaginal sleeve of FIG. 5.

FIG. 7 contains a further embodiment of a canine vaginal sleeve.

FIG. 8 contains a further embodiment of a canine vaginal sleeve.

FIG. 9 contains results of FAT tests for CHV. “Negative” means no CHV detected.

FIG. 10 contains results of FAT tests for CHV for the three of the tests with highest numerical values in FIG. 9. “Negative” means no CHV detected.

FIG. 11 contains FAT test results for CHV for all but the three highest numerical values in FIG. 9. “Negative” means no CHV detected.

FIG. 12 contains a composite number of negatives/positives for the tests of the previous figures.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

Canine herpesvirus is normally an infection of 2-5 day old puppies. Clinical systemic canine herpesvirus infections in dogs older than few days of age are rare. However, in the last several years, increasing numbers of cases of canine herpesvirus in older puppies and dogs have been observed. The present disclosure provides a much needed understanding of the role of canine herpesvirus as an emerging pathogen of dogs and its role in adult dog respiratory and systemic herpesvirus infections. The disclosure also provides novel methods of detecting and/or confirming CHV infections in individuals suspected or known to have been exposed to CHV, or suspected of having contracted the disease, and in archived formalin-fixed, paraffin embedded samples. Also provided are methods of preventing and/or managing the occurrence or spread of CHV, especially in the setting of group-housed dogs, including a vaginal sleeve to prevent CHV transmission from mother to offspring during birth.

### DETECTION OF CHV-1 IN FIXED, PARAFFIN EMBEDDED SAMPLES

As described herein, a CHV-1 specific PCR test to detect CHV-DNA in formalin-fixed and paraffin embedded tissues was designed and utilized herein. Although the stability of CHV is low in short term in fresh tissues, surprisingly the stability of the CHV DNA was found to be long term in formalin-fixed, paraffin-embedded (FFPE) tissues. This is advantageous for example, in conducting analyses of infections over time, or in different locations (e.g. over geographical areas), for example, in order to establish the status and characteristics of the spread of the virus, to assess breeding populations, and to guide selection of and assess the effectiveness of treatment methods. FFPE sections allow easy, safe and inexpensive transportation of specimens for diagnostic purposes without the need for refrigeration. Generally, the presence of CHV-1 in a sample is confirmed by detecting at least one protein that is specific for CHV-1 or at least one gene encoding a protein that is specific for CHV-1. In one aspect, the target protein for diagnostic is glycoprotein B (gB, UL27). This protein is essential for entry of the herpesvirus by fusion of the envelope with the plasma membrane. gB protein binds to heparin sulfate on the cell surface and induces neutralizing immune responses. The gB protein, and/or the gene encoding the gB protein, thus offers a suitable target for qualitative and quantitative CHV detection in samples. gB is also a sensitive target because of its high copy number. However, other CHV proteins and/or genes may also be assessed. In some embodiments, the concentration of MgCl₂ for the CHV-gB specific PCR is optimized to about 1.3-1.6 mM. In preferred embodiments, the MgCl₂ concentration is 1.5 mM.

The samples that are assayed according to this aspect of the invention are obtained from any canine subject known or suspected of having a CHV-1 infection, or for whom testing is desirable for any reason. Exemplary samples include those from any tissue and/or cells that are known to contain CHV-1 or are suspected of containing CHV-1, and which have been fixed and embedded in paraffin. Such samples include but are not limited to tissue and/or cells from: kidney, liver, lung, nasal passages, placenta, spleen, thymus, cornea, fetal tissue, vaginal tissue. In exemplary embodiments, the samples are from the kidney, liver, lung, nasal passages, placenta, spleen, or thymus. Samples may be from a cadaver or from a live donor (i.e. may be obtained ante- or post-mortem).

Various techniques may be used to “fix” a sample. One common method is formaldehyde fixation, which is typically carried our using 10% Neutral Buffered Formalin (NBF), that is approx. 3.7%-4.0% formaldehyde in phosphate buffered saline. Other aldehydes for fixation include glutaraldehyde, and combinations of formaldehyde and glutaraldehyde are also employed, as is the HEPES-glutamic acid buffer-mediated organic solvent protection effect (HOPE) method, and the like.

An exemplary fixation protocol is as follows: tissues to be fixed and processed are cut to a suitable size and thickness (e.g. 3 mm or less) and fixed by exposure to e.g. 10% formalin at room temperature, typically for about 8-24 hours. Thereafter, samples are rinsed with running tap water for, e.g. 1 hour. Alternative fixation of tissue can be carried out in a milder fixative such as zinc fixative, if desired. Water is removed from the samples by dehydration e.g. by passing the tissue through a series of increasing alcohol concentrations, e.g. 30%, 50%, 70%, 80%, 90%, 95% and 100% alcohols for about two hours each. The blocks are then placed in a 100% ethanol solution to ensure that all water is removed. Prior to paraffin embedding, the tissues are exposed to an intermediate fluid that is miscible with both ethanol and paraffin, e.g. toluene, benzene, chloroform, xylol, etc. For example, the blocks may be placed in a 50:50 mixture of absolute ethanol and toluene for two hours and then placed into pure toluene. The blocks are then exposed to a 50:50 mixture of toluene and paraffin at 56° C.-58° C. (the melting temperature of paraffin) for about one hour, and subsequently transferred to pure paraffin at 56° C.-58° C. for about one hour. Finally, samples are transferred again to fresh pure paraffin and incubated for an additional two-three hours. When placed at room temperature, the samples harden into blocks that can be stored and/or manipulated, e.g. sliced into thins section for further processing. Detecting the gB gene required removal of paraffin (e.g. by melting and/or the use of a commercial product such as BiOstic™). The retrieved material is then washed with a suitable solvent, e.g. with ethanol, and molecules of interest such as nucleic acids are extracted using known methods e.g. via a QiagenEasy protocol.

Once extracted, genetic material of interest (e.g. DNA, etc.) is detected using a suitable technique, for example, sequencing via PCR, in which primers specific for the targeted gene (e.g. gB) are used for gene amplification.

### CHV-1 VACCINE AND/OR IMMUNOGENIC COMPOSITIONS

The present invention provides attenuated CHV-1 viruses for use, e.g. in the preparation of vaccines and/or immunogenic compositions. When administered to a canine, the compositions elicit the production of antibodies against CHV-1. In some aspects, administration of the compositions elicits a protective immune response against CHV-1 in the recipient. A “protective” immune response is one which prevents the development of deleterious symptoms of disease when the recipient, at a later time, is exposed to an infectious dose of a wild-type, non-attenuated virus. Protection may be complete, i.e. no overt symptoms are observable or detectable in the vaccine recipient; alternatively, the severity and/or duration of symptoms may be lessened in an individual to a level that is less than that which would be experienced but for the administration of the composition. Efficacy of the compositions may be determined, for example, by comparing antibody titers in serum of control (un-vaccinated) vs experimental (vaccinated) subjects. For example, a ratio of about 1:4 in antibody titer of unvaccinated vs vaccinated subjects is generally taken as sufficient protection after vaccination. Vaccine efficacy can also be determined by assessing (quantitating) shedding of CHV-1 (e.g. in stool or body fluids such as nasal and genital secretions), lesion scores, Fluorescent antibody testing (FAT) antigen detection and PCR evaluation of CHV-1 DNA, etc.

Immuno-modified live virus for use in the compositions is provided by passaging a CHV-1 isolate of interest, e.g. isolate 12010974, for about 50-80 passages on Madin-Darby canine kidney (MDCK) cells to cause CHV-1 attenuation. Suitable efficacy tests, including challenge tests, are conducted to determine the amount of attenuated virus that is required to elicit an acceptable immune response. Generally, from about 10⁻³ to about 10⁻⁵ or more attenuated virus particles are used in each immunizing dose of the vaccine. In some aspects, about 10⁻⁴ virus particles are used per dose. In other aspects, the vaccine contains from about five to about six logs of virus antigen per dose.

The present invention provides compositions for use in eliciting an immune response and/or vaccinating dogs against CHV-1 infection and illness caused by the virus. The compositions include one or more types of substantially purified attenuated CHV-1 viruses as described herein, and a pharmacologically suitable carrier. The preparation of such compositions for use as vaccines is known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The liquids may be aqueous or oil-based suspensions or solutions. The active ingredients may be mixed with excipients which are pharmaceutically acceptable and compatible with the active ingredients, e.g. pharmaceutically acceptable salts. Suitable excipients are, for example, water, saline, dextrose, glycerol, ethanol and the like, or combinations thereof In addition, the composition may contain minor amounts of auxiliary substances such as wetting or emulsifying agents, pH buffering agents, and the like. In addition, the composition may contain other adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of attenuated virus in the formulations may vary. However, in general, the amount in the formulations will be from about 1-99%. Still other suitable formulations for use in the present invention can be found, for example in Remington's Pharmaceutical Sciences, Philadelphia, Pa., 19th ed. (1995).

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” refers to the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-.beta.-hydroxynaphthoates, gentisates, isethionates, di-p-toluoyltartrates, methanesulfonates, ethanesulfonates, benzenesulfonates, p-to luenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

The compositions may be administered in vivo by any suitable route including but not limited to: inoculation or injection (e.g. intravenous, intraperitoneal, intramuscular, subcutaneous, and the like). Other suitable means include but are not limited to: inhalation (e.g. as a mist or spray), orally (e.g. as a pill, capsule, liquid, etc.), by ingestion of a food or probiotic product containing the attenuated virus, etc. In preferred embodiments, the mode of administration intramuscular or subcutaneous. In addition, the compositions may be administered in conjunction with other treatment modalities such as substances that boost the immune system, various chemotherapeutic agents, other antigenic agents (e.g. a part of a combination vaccine), various adjuvants, and the like.

In yet other aspects, the virus that is utilized in the compositions is a killed or inactivated virus. Vaccines of this type are created by inactivating a pathogen, typically using heat or chemicals such as formaldehyde, formalin or binary ethylenimine (BEI). This destroys the pathogen's ability to replicate, but keeps it “intact” so that the immune system can still recognize it. The selection of a viral strain for inactivation and administration in this aspect is similar to that of selecting a live virus for attenuation, e.g. one or more of the virus isolates disclosed herein is used, and the compositions for administration are formulated in a similar manner.

In yet other aspects of the invention, what is provided is a method of vaccinating a subject in need thereof against CHV-1 infection. The method involves administering an efficacious amount of a composition comprising an attenuated or killed virus as described herein. The efficacious amount is sufficient to prevent the development of or lessen the extent of the development of symptoms of CHV-1 infection in the subject, when the subject is later exposed to wild-type CHV-1 or contracts a CHV-1 infection. Descriptions of exemplary compositions and methods of administration are provided above. The subject to whom the composition is administered is generally a canine, for example, a domestic dog or other susceptible wild carnivores such as foxes, wolves, and coyotes. In some aspects, the subject to whom the composition is administered is a puppy of an age ranging from about 1 day to about 1 month in age, although older puppies and dogs may also benefit from receipt of the vaccine. Generally, an initial dose is given before the puppy is 1 month old or up to 6 weeks old, and thereafter, booster doses are administered e.g. at 6 weeks, 9 weeks, 12 weeks, and/or annually. Similar doses may administered for older dogs, e.g. >1-year old.

According to another embodiment, individual and/or herd immunity is built in dogs housed in groups by administration of an attenuated or a killed CHV-1 vaccine, as described herein. This is accomplished by administering the vaccine to dogs residing at the location, e.g. to one or more of puppies, juveniles and adults. In some aspects, the appropriate vaccine type (killed or attenuated) for use is selected based on the pregnancy status of the animal. Generally, non-pregnant animals receive modified live virus vaccine and pregnant vaccine receive killed virus vaccine.

In some embodiments, the vaccine of the invention is administered with one or more additional canine vaccines such as a vaccine for canine parvovirus, canine distemper, canine adenovirus, canine parainfluenza virus, and canine coronavirus.

### VAGINAL DOUCHE FOR TREATMENT AND PREVENTION OF CHV-1 IN FEMALE DOGS

In some aspects, the present invention provides methods of preventing transmission of CHV-1 from female dogs that are shedding the virus to offspring during birth or to other dogs by contact with vaginal secretions. The method involves administering, to a female dog, a vaginal douche comprising an anti-herpesvirus compound. In some aspects, the female is known to carry CHV-1, e.g. she is shedding virus. Contact between CHV-1 residing in the vagina of the dog results in killing of the virus, thereby decreasing or eliminating the viral load, and decreasing or preventing transmission of the virus to off-spring during birth, or to other dogs via direct contact or via contact with shed virus. Exemplary anti-herpes compounds that may be used to formulate the compositions include but are not limited to: acyclovir, acyclovir elaidate, fencyclovir, idoxuridine, cytarabine, vidarabine, trifluorothymidine, 9-8-D-arabinofuranosyl-2,6-diaminopurine (ara-DAP).

Administration of the douche is typically carried out when a female dog is known or suspected of being infected with CHV-1, e.g. using a detection method as described herein. Use of the method prevents vaginal shedding of CHV-1 and thus prevents transmission of the virus to dogs that come into contact with the infected female, or who come into contact with vaginal discharge of the female. Significantly, transmission to puppies during birthing (e.g. as they travel through the vagina) is also prevented.

### CHV-1 INTERVENTION AND CONTROL STRATEGIES

According to another embodiment, there is provided an intervention strategy to prevent the transmission of CHV-1 infection within group-housed dogs, and/or between groups of group-housed dogs, such as in and/or between breeding kennels. One such strategy would be implemented by carrying out one or more of the following:
The dogs are prescreened before introduction to breeding kennels based on CHV-1 SN test;
The animals are also screened by detection of shed virus in a pooled sample of secretions from external epithelial surfaces or orifices (e.g. a pooled sample of vaginal or nasal secretions, saliva, conjunctiva, etc.) to ensure they are not shedding the CHV-1;
The animals can be periodically monitored at regular intervals, e.g. once per year, by detection of a CHV-1 specific protein or a gene encoding a CHV-1 specific protein (e.g. by PCR) of a pool of external epithelial surfaces as described here;

According to a further embodiment, there is provided a system of reducing or eliminating the risk of introduction of CHV-1 into enzootic areas by periodic disinfection with 0.01% bleach of floors, and avoiding contact with wildlife such as foxes, wolves, and coyotes.

### VAGINAL SLEEVE

When a pregnant female is infected with a vaginally transmissible disease (e.g. a disease caused by a virus, bacteria, fungus, etc. that is present in vaginal secretions), offspring can contract the disease by exposure to the infectious agent while traversing the birth canal. To address this problem, the present invention provides vaginal sleeves which protect offspring during birth. The vaginal sleeve allows puppies to be delivered by natural processes without the need for caesarian section. The sleeves may be disposable and are easy to use. While originally conceived for use in canines to prevent CHV-1 transmission, the devices can be tailored for use in any mammalian species, including humans, to protect against transmission of any vaginally transmissible disease. The discussion below focuses on canines but many of the general principles apply to the use of the sleeves in any mammalian species.

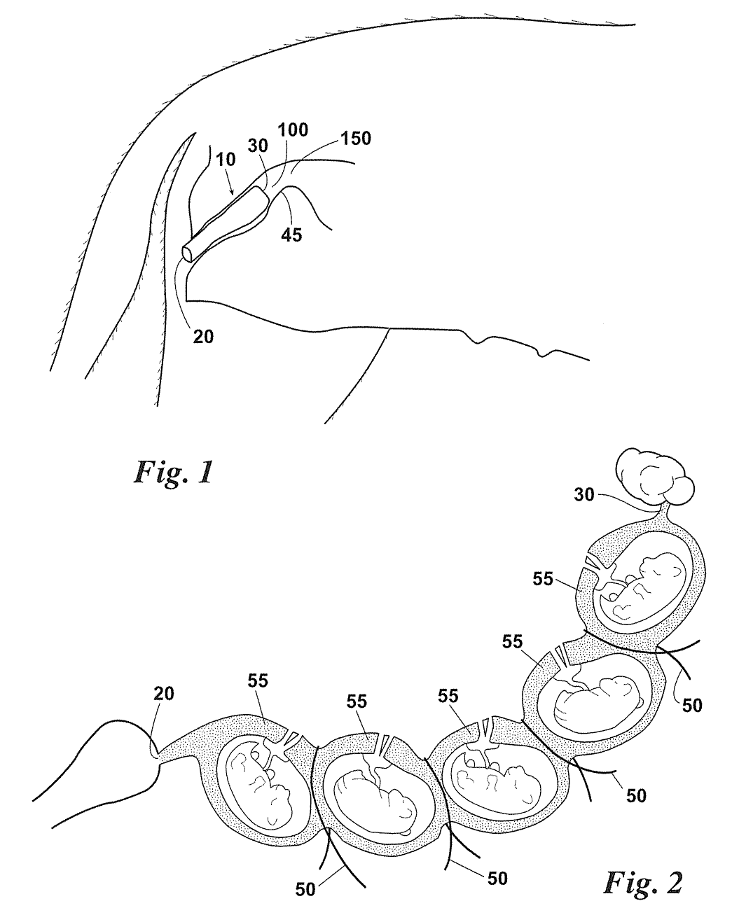

In one aspect, the devices are use during the delivery of puppies from CHV-1 positive female dogs. When used, a sleeve is inserted (e.g. using a speculum or other comparable device) into the vagina of a whelping female and rests against the vaginal walls that are closest to the uterus (and may also occlude cervical tissue that is exposed within the vagina). During birth, a puppy passes through the cervix and then, rather than contacting the vaginal walls directly, instead enters the sleeve and traverses the birth canal while remaining inside the sleeve. This is illustrated schematically in FIG. 1, which shows vaginal sleeve 10 with proximal end 20 and distal end 30 inserted into uterus 100. Distal end 30 is positioned against the upper end of the vaginal wall 45, which contains cervical opening 150 through which puppies (not shown) will emerge from uterus. Several different embodiments of vaginal sleeves are described herein.

Generally, as illustrated in FIG. 5, vaginal sleeve 10 has the shape of a tube or channel that is roughly or substantially cylindrical and comprises an opening at both the proximal and distal ends, 20 and 30, respectively. The sleeves are flexible, allowing propulsion of the contents by manual manipulation, and are generally transparent or semi-transparent, permitting visual observation of the contents, i.e. the intra luminal contents of the device sleeve are visible after the fetus is expelled from the uterus. The sleeves are of a size and have dimensions suitable for being fitted closely into the vaginal canal of a female dog that is about to give birth, making contact with and covering the vaginal walls. The size and dimensions of the sleeves can be tailored for specific breeds and/or specific sizes of dogs.

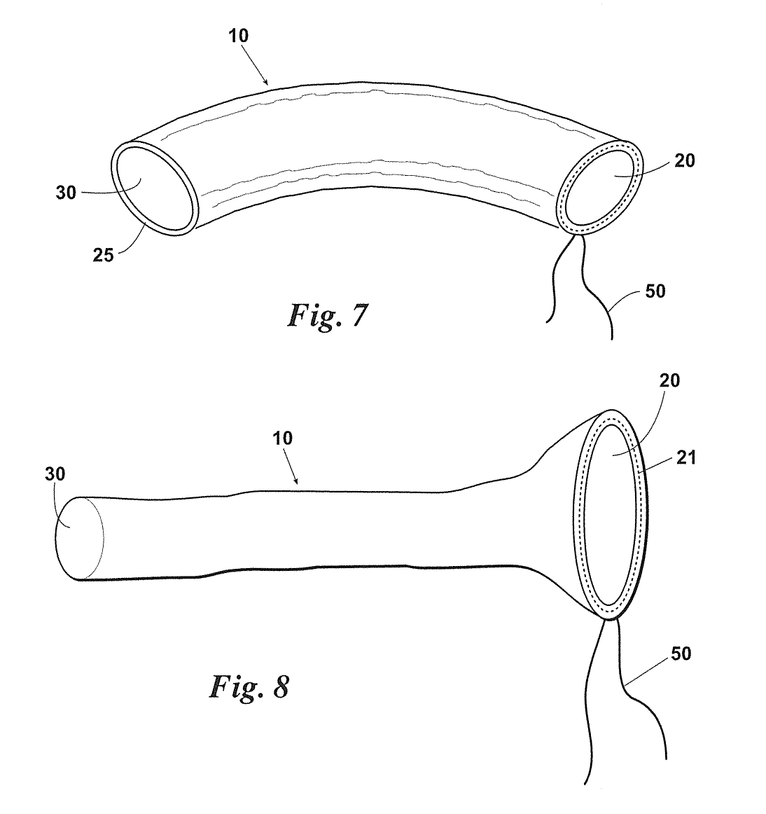

A distal end of the sleeve is inserted into the vagina and makes direct contact with the upper end (back) of the vaginal canal and/or with the cervix. In some aspects, the distal end of the sleeve is substantially circular and fits against the cervix, the walls of which are generally thinned and the opening of which is dilated in preparation for birth. The distal end of the sleeve blocks or occludes as much of the tissue at the end of the vagina as possible, including exposed cervical tissue, to prevent contact between the offspring and tissues of the mother which harbor an infectious agent. Generally, the distal end of the sleeve comprises a mechanism for retaining its shape during use, i.e. it is a rigid or semi-rigid ring (e.g. ring 25 of FIG. 7) that is resilient so that it stays in place and open after insertion into the vagina, allowing a puppy to enter the sleeve as it emerges through the cervical opening and into the sleeve. For example, a somewhat rigid but still deformable and flexible ring such as those used in some condoms may be employed. The distal end of the sleeve that contacts the upper vaginal wall and cervix may be or may comprise a flattened ring, e.g. of cushioned or spongy material to provide good contact with the tissue. The general shape of the distal opening is maintained or recovered after a puppy passes through the opening and proceeds into and down the central channel of the sleeve, eventually exiting the vagina. Because the puppy is within the sleeve, direct contact between the vaginal walls and the puppy is prevented and CHV-1 (or another disease-causing agent) cannot be passed from the mother to the puppy during vaginal transit. Alternatively, or in addition, as shown in FIG. 5 and in detail in FIG. 6, distal end 30 may comprise a telescoping ring 60 with outer segment 61 and inner segment 62 which fits slidably within outer segment 61. Sliding more of inner segment 62 into outer segment 61 makes the total circumference of adjustable ring smaller, which sliding inner segment 62 out from outer segment 61 increases the total circumference of adjustable ring 60. Ring size is thus adjustable so that the circumference of the ring can be adapted as needed to fit a particular subject. In some aspects, the proximal end of the sleeve (the end that permits egress of the puppy from the vagina and into the outside environment) also comprises an optional mechanism that is similar or comparable in nature to that which is in the distal end, so that the proximal end of the sleeve also stays open after insertion of the sleeve into the vagina.

In some aspects, such as that shown in FIG. 8, proximal end 20 of vaginal sleeve 10 is flared i.e. of greater circumference than the rest of the sleeve, e.g. in order to allow access to the puppy by a birthing assistant such as a veterinarian.

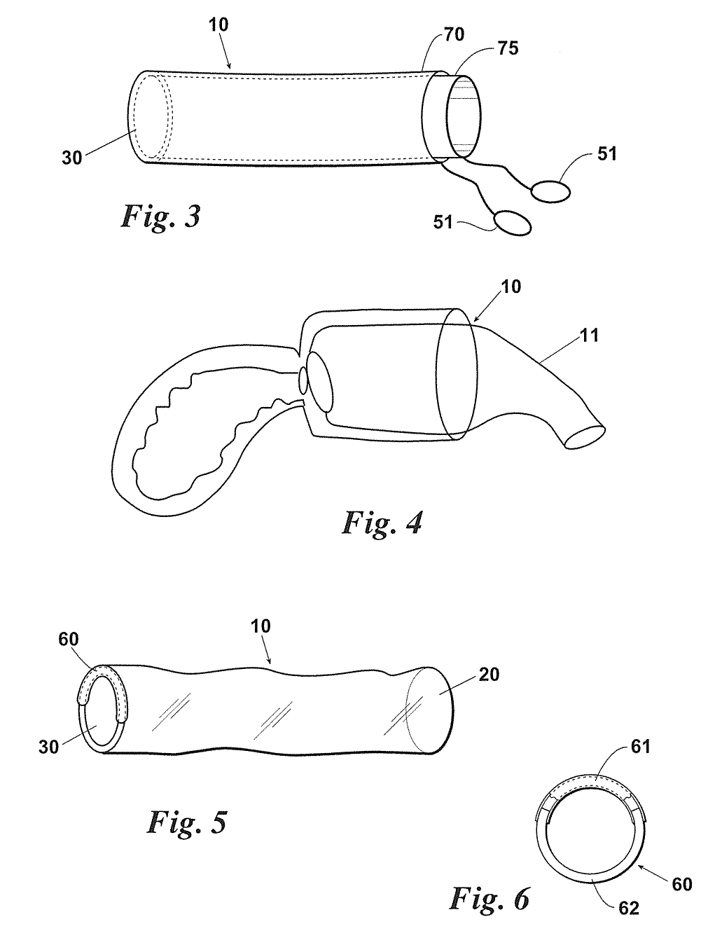

In further aspects, illustrated in FIGS. 7 and 8, proximal end 20 of vaginal sleeve 10 also comprise a “purse string” 50 or other mechanism (e.g. a string, tab, strip, etc., illustrated in FIG. 3 as component 51) which can be used to remove the sleeve from the vagina after use, and/or alternatively to close the proximal end of the sleeve. In this aspect, purse string 50 is threaded through channel 21 at proximal end 20 of vaginal sleeve 10 and two ends of purse string 50 are exposed. Channel 21 may be formed by folding back and securing the material from which the sleeve is made, or by attaching material to the outer or inner surface of the edge of proximal end 20 to form the channel. Pulling on the two ends of purse string 50 tightens purse string 50 within channel 21, decreasing and/or altogether closing proximal end 20. With this type of “purse string” closure, once a puppy is ensconced in the sleeve, the proximal end can be closed and the sleeve can be removed (e.g. by pulling the vaginal sleeve 10 out of the vagina via the ends of purse string 50) with the puppy contained therein. In fact, in this embodiment, the proximal end need not be open during the birthing procedure. The puppy is then released from the sleeve e.g. after disinfection of the outer surface of the sleeve, by, for example, carefully rolling the sleeve back on itself to expose and remove the puppy without contact between the puppy and the outer surface of the sleeve, which may have been contaminated by direct contact with the vaginal walls.

Dogs typically produce more than one puppy per birth event (a “litter” of puppies). In some aspects, one sleeve may be used for the delivery of one puppy. In this embodiment, once a puppy has entered the sleeve, the device can be removed from the vagina of the dog, facilitating the puppy's exit from the birth canal, and a new sleeve is inserted to receive subsequent puppies. Alternatively, more than one puppy is birthed through the same sleeve, with each puppy of the plurality of puppies emerging from the proximal end of the sleeve and into the environment one after the other.

In yet another aspect, illustrated in FIG. 2, a single sleeve comprises a plurality of purse strings 50, two of which define a separate section or compartment 55 of vaginal sleeve 10. The compartments are thus separated from one another by purse strings 50, which are open prior to delivery. In this embodiment, a proximal portion of the sleeve that is so divided into sections protrudes from the vagina after insertion of the sleeve. Multiple puppies enter and traverse the sleeve one after another, and when the first puppy emerges from the vaginal canal, it is guided to the proximal-most compartment of the sleeve and the purse string mechanism is used to close that section off from the rest of the sleeve. As a second puppy emerges, it is guided into the next available section, which is then closed off via closure of a purse string, etc. In this way, multiple puppies are each delivered into separate compartments of a single sleeve. The sleeve and puppies are removed after birthing is complete. It is advantageous to restrain from cutting or removing the umbilical cord until the puppy has been removed from the birthing canal and the vaginal sleeve.

In yet other embodiments of the device of the invention, illustrated in FIG. 3, the vaginal sleeve has two parts: outer portion 70, the outer surface of which makes direct contact with the vaginal walls, and inner liner 75, the outer surface of which makes direct contact with the inside surface of outer portion 70 and the inner surface of which makes direct contact with a puppy. In other words, outer portion 70 houses inner liner 75 and the sleeve is “double walled”. In this embodiment, the entire vaginal sleeve 10 comprising both portions is inserted prior to birthing, and outer portion 70 (which may be made of somewhat more rigid material than inner liner 75) remains in place during the entire birthing process. Inner liner 75 may comprise a component 51 attached thereto, which may be a purse string as described above, or may be another device such as a tab, strip, flap, etc. that does not function to close the sleeve as a purse string does, but rather as a device to remove the sleeve by pulling thereon. In some embodiments, outer portion 70 is then reloaded with a fresh inner sleeve 75 which receives the next puppy; alternatively, two or more puppies can be received within one inner liner 75, or inner liner 75 may comprise multiple compartments separated by purse strings as described above. In this latter aspect, outer portion 70 is removed after all puppies have been born and may be disposable, or may be sterilized and reused.

In another embodiment, illustrated in FIG. 4, vaginal sleeve 10 is elongated and protrudes from the vagina, e.g. by a narrowed neck-like portion 11. This embodiment serves to illustrate only one of the many design variations that will occur to those of skill in the art, and all such embodiments are encompassed by the present invention.

Those of skill in the art will recognize that a source of light will generally be provided for use with the vaginal sleeves during the birth process. The source of light can be hand held or attached to the head of the operator, but is not part of the sleeve device itself The source of light may be needed, e.g. for intra-vaginal observations during birth.

The body or tubular section of the sleeve may be formed from any of a wide variety of suitable materials. The selected material is generally soft, flexible and may be somewhat “stretchy” or elastic or expandable, but is generally resilient and retains or returns to its original shape after deformation (e.g. by passage of a puppy through the sleeve). The material is generally fluid (e.g. water) impermeable (“waterproof”) but may be gas (e.g. O₂) permeable. The material may be transparent or partially transparent or may be opaque. Certain advantages may be had by using transparent materials or materials which allow at least some visibility of the contents e.g. so that the progress of a puppy through the sleeve may be observed. The material is generally suitable for sterilization. Those of skill in the art will recognize that, for a two-part device comprising an outer portion and an inner liner, the outer portion may be more rigid or less flexible than the inner lining portion since the outer portion is designed to remain within the channel during the entire birthing procedure and to support the inner lining and the puppy, but need not actually accommodate the contours of a puppy, as the inner lining must generally do.

Exemplary materials that are used include but are not limited to: plastics such as polyvinyl chloride, polyethylene, polypropylene, polyurethane, latex, various rubber polymeric materials, etc. Various rubber latexes with reinforcing agents such as those described in U.S. Pat. No. 8,017,680 may be used, as may the materials disclosed in published US patent application 2002/0038658. The tube portion of the vaginal sleeve need not be formed from the same material as e.g. the distal ring, and/or the tube portion of the vaginal sleeve need not be formed from the same material throughout. For example, segments or bands of the sleeve may be reinforced with multiple layers of material or a thicker layer of material, or formed from a stronger, heavier weight material, than is the majority of the tube. For example, if purse strings are present along the tube, segments of the tube which contain the strings and channels to accommodate the strings (e.g. segments which are collapsed when the tube is closed by pulling the string taut) may be reinforced.

In general, the vaginal sleeves and inner liners described herein are disposable. However, they are comprised of materials that are sterilizable and may thus are sterilized before use, and may be sterilized and reused. The sleeves may be sterilized by heat, x-rays, and/or exposure to chemicals such as disinfects, alcohols, etc. One or more anti-herpes agents may be used to treat the sleeves before use, and in some embodiments, the anti-herpes agents remain on or in the sleeves before use, e.g. as a coating, or impregnated into the material from which the sleeve is made. Further, the inner and/or outer surfaces of the sleeves may be lubricated prior to use to promote easy insertion into the vagina, and to facilitate easy ingress of a puppy into the sleeve.

### EXAMPLES

### Abstract

This example describes canine herpesvirus (CHV-1) infections circulating in a large breeding kennel of English Bull dogs with clinical disease, death and lesions in older dogs (9 weeks of age) in South-Central USA. The CHV-1 virus has been repeatedly detected in the kennel in several animals for at least 3 years based on clinical signs and confirmation of CHV-1 infections based on virus detection using virus isolation and direct CHV fluorescent antibody test, antibody detection based on CHV-serum neutralization, virus DNA detection using CHV specific-PCR targeted to glycoprotein B (gB) of CHV, and histopathology lesions compatible with CHV. Using CHV gB specific PCR, we were able to detect CHV DNA in archived paraffin-embedded, formalin-fixed tissue blocks. Several forms of the CHV-1 related syndromes were observed in the kennel including acute fatality in young and older puppies, respiratory disease, abortions, fetal reabsorption, mummification, corneal ulcers in adult dogs over 5 years of age and conjunctivitis in young puppies. Transfer of immune plasma (20 ml per puppy, SQ, with CHV titer above 1:32 by CHV SN test) from herpes virus exposed adult females has offered symptomatic relief and prevented mortality in newborn naïve puppies compared to untreated puppies. Because immune untreated plasma has shown clinical protection, we conclude that availability and application of commercial vaccine may provide relief from clinical diseases associated with CHV-1 infections in the USA. Based on the studies using direct fluorescent study in ante-mortem epithelial samples in animal shelters, we conclude that canine herpes virus associated diseases are quite wide-spread in the USA.

In this study, we validated and applied two methods for rapid, sensitive and specific detection of CHV-1 in antemortem vaginal epithelial specimens and also archived paraffin-embedded tissue blocks from kennels with ongoing CHV-associated problems. Due to ongoing CHV problems, management approaches were applied to prevent transmission of CHV to susceptible naïve newborn animals and immunotherapy with CHV immune plasma therapy to prevent mortality. This study provides specific diagnostic and management approaches applied on populations in kennels.

### Introduction

Canine herpesvirus (CHV-1) is an alpha herpesvirus that is well documented to infect newborn puppies. The infection is transmitted by contact with secretions and excretions of the carrier dogs housed in the groups (1). Although, CHV-1 is widespread in canine populations based on serum antibody prevalence in different countries (36% to as high as 94%) in the Western hemisphere (1). The role of CHV in canine disease is still not completely understood because large scale population studies have not been conducted. In a recent study Germany, CHV was not detected in sick and healthy dogs with respiratory disease using polymerase gene as target for PCR (2). However, selection of target for PCR can make a difference in the sensitivity of the PCR. Moreover, CHV-1 circulates in animal shelters due to exposure of naïve animals to dogs shedding CHV-1 following close contact between animals kept in groups (3). Three forms of diseases due to CHV-1 have been documented: reproductive disease with abortions and stillbirths, respiratory and conjunctivitis. All these CHV syndromes are caused by a virus that has one serotype and is closely related genetically, worldwide (4). These epidemiological studies cannot not distinguish between continuous circulation of the same virus and/or frequent reintroduction of new CHV isolate. Based on this study, CHV problems are widespread in the American dogs that are housed in groups. However, the diagnostic methods such as histopathology for CHV-1 detection lack sensitivity. In this study, we validated and applied two methods for rapid, sensitive and specific detection of CHV-1 in antemortem vaginal epithelial specimens and also archived paraffin-embedded tissue blocks from kennels with ongoing CHV-associated problems. Due to ongoing CHV problems, management approaches were applied to prevent transmission of CHV to susceptible naïve newborn animals and immunotherapy with CHV immune serum to prevent mortality. This study provides specific diagnostic and management approaches applied on populations in kennels.

### MATERIALS AND METHODS

Clinical Specimens from the USA: According to one example, a total of 434 specimens have been examined for canine herpesvirus. These included fresh specimens such as kidney (n=56); liver (n=60); lung (n=147); nasal specimens (n=12); placenta (n=2); spleen (n=6); thymus (n=4) and miscellaneous specimens from the USA.

Direct fluorescent antibody test for canine herpesvirus: For rapid and accurate confirmation of canine herpesvirus infection in the fresh tissues according to the exemplary embodiment, a direct fluorescent antibody test was performed with anti-CHV-1 polyclonal antiserum FITC conjugated of canine origin (VMRD, Pullman, Wash.). Briefly, sections of the liver and kidney were fixed with acetone-methanol at room temperature for 15 minutes. The sections were incubated with CHV-1 FITC conjugate for 30 minutes. After rinsing the unbound conjugate, the sections were counter stained with Evans blue.

To prepare the ante-mortem spot slides from external epithelial surfaces in this embodiment, swabs were collected from vagina, conjunctiva, nasal and prepuce of dogs. The swabs were vortexed to release the epithelial cells. After centrifugation, the cell pellet was deposited on charged spot slides (8-spot slides). After the cells were attached, the epithelial cells were fixed and stained as described above.

Virus Isolation: For isolating the CHV-1 in one embodiment an approximately 10% (w/v) suspension of puppy tissues was prepared in virus transport medium. The fresh chilled tissues were homogenized and divided in smaller pieces with scissors in a 50 ml tube. After vortexing for 2 minutes, the suspension was centrifuged for 3000 rpm for 15 minutes to pellet the tissue. The clear supernatant was syringe filtered through a 0.22 μm filter (GE Healthcare UK, Buckinghamshire, UK). About 1 ml of tissue suspension was inoculated on Madin-Darby canine kidney cell line that was plated to form a semi-confluent monolayer (80%) on day 2. The cells were observed daily for 1 week for herpesvirus cytopathology with rounding of cells and detachment of the cells.

Total DNA Extraction from Archived Cases: For investigating the detection of canine herpesvirus DNA in formalin-fixed sections from archived cases, one embodiment uses 4 micron thick sections. The sections were treated with 1 ml of BiOstic (MOBIO laboratories, Carlsbad, Calif.) to remove paraffin. After washing with ethanol, the tissue section material was air-dried. The section material total DNA was extracted by QiagenEasy protocol (Qiagen).

Canine Herpesvirus Glycoprotein B (gB) Specific PCR: Continuing with the present example, the CHV-gB specific PCR was performed using MgCl₂ (1.5 mM), Amplitaq Gold 10× buffer (Applied Biosystems), CHV-gB forward and reverse primers (15 uM), dNTPs (10 mM each), and AmpliTaq Gold (5U/ul). The PCR procedure was optimized and final PCR product of about 120 by was detected. The identity of the amplicon was confirmed by sequencing and BLASTn analysis. The forward primer sequence was 5′-caggactattggactatagt-3′; and reverse primer was 5′-ttgcaatgcccctcataatt-3′. The PCR program used was as follows: denaturation at 94° C. for 10 minutes; and 35 cycles of 94° C. for 30 seconds, 50° C. for 1 minute, and extension of 72 for 1 minute. Final PCR extension was 72 for 10 minutes and reaction was held at 4° C. The PCR products were electrophoresed on 2% agarose gel and the size of the amplicon was 120 bp.

The PCR was found to be specific for detection of CHV infection in archived tissue blocks because this gene has high copy number. The validity of the CHV was confirmed using a CHV isolate and the conditions for PCR were optimized. The PCR products were gel-purified and sequenced. The PCR amplicon was found to have maximum homology with the gB gene of canine herpesvirus on BLASTn analysis. All nine out of 9 known CHV positive paraffin-embedded, formalin-fixed tissues were PCR positive after 3 years of storage.

### RESULTS

Clinical cases and specimen types from the English bulldog kennel, AR:

A total of 4 whole body necropsies were performed in a period of 3 years. A total of eleven antemortem swabs (vaginal swabs) from periwhelping female dogs (in the last week of pregnancy) were examined for canine herpesvirus. A total of 17 sera were examined by CHV-SN at the Cornell Animal Diagnostic Laboratory, Ithaca, N.Y.

Necropsy Case # 12010974:

The kennel owners of a large kennel of English Bull dogs (n=57 with females=53 and males=4) with a history of canine herpesvirus infection purchased a litter of puppies and introduced them to their breeding population. All puppies were normal at first but ten days after introduction a 9-week old female dog in good health was reported to suddenly become weak and hypothermic. Death followed rapidly. The referral veterinarian marked the case “atypical herpesvirus” due the age of affected puppy (9 weeks). The corpse was refrigerated and sent for necropsy and confirmation of the cause of death and fresh tissues (lungs, liver, and kidney) were submitted for virology testing.

A diagnosis of CHV-1 was confirmed by isolation in Madin-Darby canine kidney (MDCK) cell line, postmortem detection of CHV-1 in tissues, and the presence of herpesvirus lesions, as described below.

During necropsy examination, petechial hemorrhages were observed on the cortical surfaces of both kidneys. The lungs were edematous and contained petechial hemorrhages. Microscopic examination confirmed hemorrhages in the kidneys and lung, accompanied by small deposits of fibrin in the lung. Hemorrhage in the kidney and lung parenchyma was centered on discrete foci of necrosis, occasionally accompanied by small deposits of fibrin. Necrotic foci were scattered throughout the liver and spleen as well. Rare numbers of intranuclear inclusion bodies consistent with herpesvirus were detected in the necrotic areas in the kidney, lung and liver. No microscopic lesions were identified in the small intestine, pancreas or colon of this puppy.

Upon fluorescent antibody test (FAT) for CHV-1, several irregular focal areas of canine herpesvirus antigen were observed. Most of the herpesvirus antigen positive cells were present between the nephrons (interstitial cells of the kidney). The kidney sections were recorded as suspect positive for canine herpesvirus due to weak positive fluorescence, likely because there was a time gap between death of the puppy and examination. This was an unusual finding because the nephrons were totally spared and negative. Lungs and liver samples were also negative. This indirectly indicated that a novel strain/genotype of CHV-1 with a unique phenotype and tropism was circulating in the kennel.

For further confirmation of CHV-1 and continuing with the present example, a pool of fresh tissues was inoculated on Madin-Darby canine kidney (MDCK) cell line treated with dexamethasone (5 micro-gram per milliliter). Based on characteristic cytopathic effects (e.g. rounding, ballooning and detachment), a herpesvirus was suspected. The virus isolate was further confirmed positive by direct fluorescent antibody staining with characteristic nuclear and cytoplasmic fluorescence with direct CHV-1 FAT.

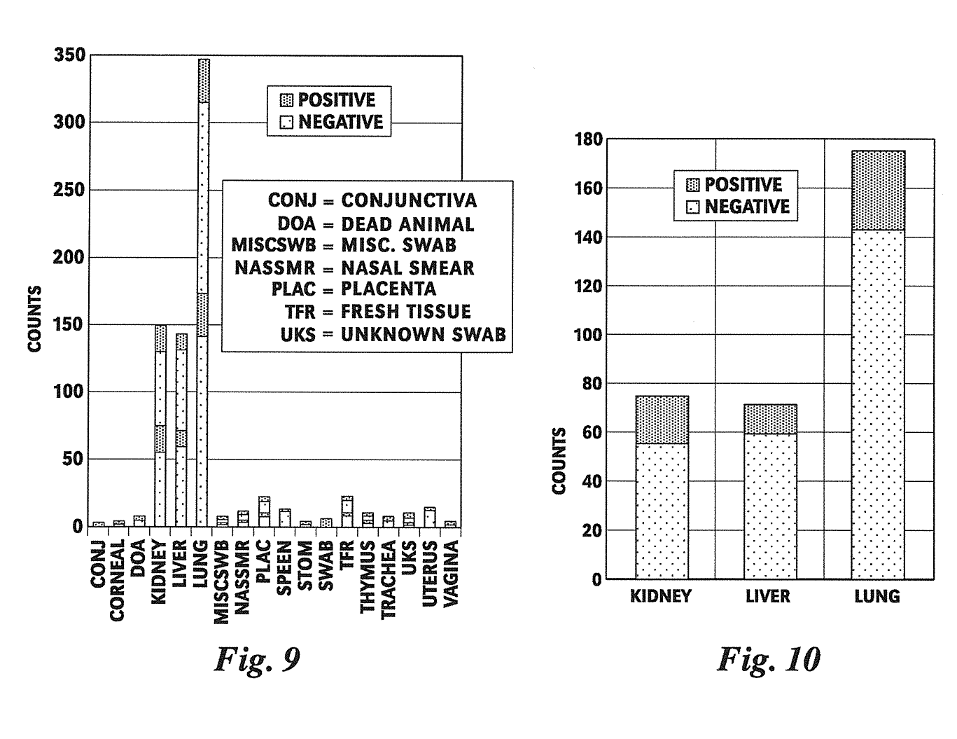

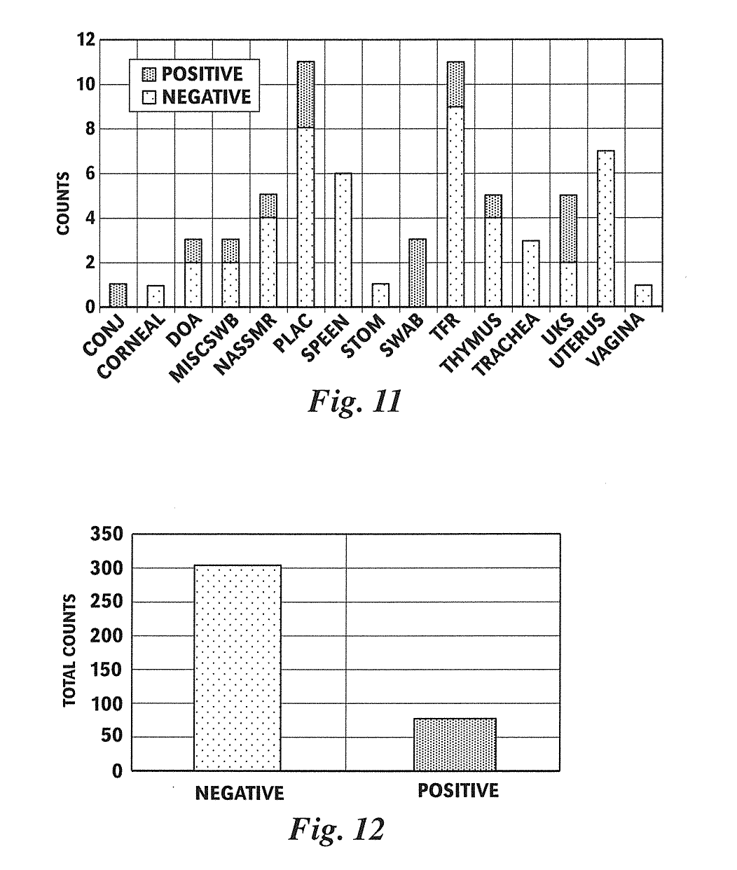

FIGS. 9 through 12 contain FAT test results for CHV for different sample acquisition sites. In FIGS. 9-12, the following abbreviations are used: Conj=Conjunctiva, DOA=Dead animal, KID=Kidney, MISCSWB=Miscellaneous swab, NASSMR=Nasal smear, PLAC=Placenta, SWB=Swab, TFR=Fresh tissue, THYM=Thymus, UKS=Unknown swab, VAG=Vagina. FIG. 12 shows the total positive/negative counts for all samples.

Five archived paraffin-embedded tissue blocks from tissue from this puppy were later examined by PCR designed using the gB gene of canine herpesvirus as the PCR target. All tissues (Block 1: Liver; Block 2: Lung and spleen tested together; Block 3 and 4: Kidneys; Block 5: Small intestine, pancreas, and colon) examined were found to positive for canine herpesvirus by CHV gB PCR. The presence of CHV DNA in these samples supports a finding of systemic CHV infection in the subject puppy.

To understand the circulation of the CHV-1 in the English bull dog kennel, a relevant database was checked and it was found that this breeding kennel reported four additional suspected cases of CHV-1 associated with abortions and respiratory disease in about 6 week old puppies after the present case was diagnosed. Further, the attending veterinarian for the kennel reported that the kennel continues to have intermittent CHV-1 related problems.

Necropsy Case # 13121323: In a second submission, a 4.5 week old puppy in the same kennel developed respiratory disease and died within 48 hours. On gross examination, necrotic foci were observed in liver, lung and kidneys. Based on pathology lesions, the pathologist concluded that necrotic foci are pathognomonic of CHV-1 infection. The abdominal and thoracic cavities contained 20 ml of dark red fluid. On histopathology examination, discrete foci of necrosis were scattered throughout the liver, lung, and renal cortex. In the lungs, the necrotic foci were accompanied by small deposits of fibrin, edema, and alveolar histocytic infiltrates. Necrosis was more severe in lung. On direct fluorescent antibody examination, the liver was negative and lung was found to be suspect for CHV-1 due to weak fluorescence and autolysis of the tissues due to proteolytic degradation of the canine herpesvirus. The presence of CHV-1 DNA was confirmed by PCR targeted to gB gene of CHV-1 in 4 stored paraffin-embedded tissue blocks (Block 1: Small intestine, mesenteric Lymph node, and colon; Block 2: Small intestine, mesenteric Lymph node and colon, Block 3: Liver, lung and kidney; and Block 4: Heart, spleen, thymus, and urinary bladder). The PCR amplicon was sequenced and confirmed as gB of CHV.

Necropsy Case # 14021368: In the third case submitted, two mummified fetuses were submitted. The lung, liver and kidney of the mummies were negative for CHV by direct FAT. It was concluded that the virus was degraded during autolysis of the tissues during storage before submission.

Necropsy Case# 14031493: In the fourth case, a 4-week old puppy was submitted. On gross examination, the internal tissues had pinpoint hemorrhages on the kidney. On the direct fluorescent antibody examination for CHV-1, the tissues were weakly positive for CHV-1.

Case # 1012549: During investigation of the case records at a veterinary hospital, it was noted that a previous submission from the same kennel to had been deemed canine herpesvirus positive by general herpesvirus PCR.

Canine Herpesvirus Serum neutralization (SN) Test: The client submitted several sera (n=17) for CHV-SN. This kennel does not use any CHV-1 vaccines because CHV vaccines are not commercially available in the USA. The CHV-SN titers on these individual canine sera were 1:24, 1:24, 1:16, 1:48, 1:96, 1:128, 1:128, 1:128, 1:48,1:128, 1:96, 1:64, 1:64, 1:48, 1:24, 1:3 and <1:2. Based on the serological prevalence, it was concluded that all dogs tested, except one, in this kennel had been exposed to CHV. In spite of the detection of high titers of serum antibodies in kennel residents, clinical problems have persisted in newborn puppies that are immunologically naïve, likely due to shedding of the CHV-1 virus days before whelping.

Direct Fluorescent Antibody Test for CHV on Vaginal Swabs from Pregnant Dogs: Eleven vaginal swabs from pregnant dogs were examined for canine herpesvirus by direct fluorescent antibody test about one week before whelping. Seven animals were negative for CHV-1 but three animals were actively shedding CHV virus at this time. One specimen had insufficient cells and was found to be unsuitable for testing by FAT.

### DISCUSSION

In the US, canine herpesvirus-1 is quite common in canine populations based on serum antibody data. However, there are no published reports on shedding of CHV and distribution of CHV in dogs that are housed in groups in breeding kennels and shelters. In fact, it is likely that the detection of canine herpesvirus is underestimated in the US because most veterinary diagnostic laboratories are not properly equipped with sensitive and specific CHV detection techniques. Also, CHV-1 is difficult to confirm due to a lack of stability of the virus in stored fresh tissues: enveloped viruses such as CHV are known to be fragile (5). PCR on formalin-fixed paraffin-embedded tissues is in general a more sensitive technique for detection of herpesviruses than is immunohistochemistry (6).

There are only few published reports of fatal systemic infections with CHV-1 in older dogs. In one recent serious outbreak in Japan with fatality the dogs were predisposed by immunosuppressive therapy for cancer (7). CHV-1 was the only pathogen identified in this fatal infectious tracheobronchitis outbreak in dogs stressed in shelter environment (3). In another case report from the US, disseminated CHV-1 infection was diagnosed in an immunocompromised adult dog (8). Except for these few isolated reports of fatal in immunosuppressed dogs, CHV-1 has not been reported in the literature in older dogs (over newborn age). There are no reports of diagnosis and management of dogs housed in groups (population studies).

In the present case, a fatal systemic infection in a nine week old dog was diagnosed after its introduction to a breeding dog facility. Problems related to CHV-1 have persisted in this kennel. Dog breeding is done year round; however, more problems have occurred in this kennel in winter months (December-February). On inquiry, the kennel ambient temperature is set at 70F. The floors are concrete with wood shavings. Puppies may have been predisposed to CHV due to hypothermia and effects of cooler ambient temperature on the young puppies predisposing to effects of CHV-1 (9). In this kennel, CHV-1 was associated with fatal systemic disease with lesions, respiratory symptoms, corneal ulcers, conjunctivitis and mummification. The problem was introduced in this naïve kennel from introduction of a CHV-1 shedder and carriers. The genetic variation among CHV-1 isolates is minimum, world-wide. Thus, it is difficult to genetically determine if the CHV-1 is due to recirculation or continuous reintroduction from newly introduced shedders. However, transplacental transfer of CHV could not be demonstrated in this particular kennel (n=1, placenta from a female animal that was positive in vagina for CHV-1 was negative for CHV-1 by FAT test on the fresh placenta sample). This observation is critical because no evidence of trans-placental CHV-1 transfer was found in pregnant dogs. Thus, a critical invention point would be to avoid vaginal delivery by performing Caesarean section in the English bull dogs.

There are no previous reports of longitudinal effects of CHV-1 after introduction in a breeding kennel. Based on this kennel and previous submissions of CHV-1 from kennels, it is appears that after CHV-1 is introduced the virus continues to circulate due to contact with CHV shedders and vaginal shedding at birth exposing the naïve puppies that are susceptible to CHV. This can lead to continued CHV-1 problems in a breeding kennel. English bull dogs and French bull dogs appear to be more susceptible to health problems. This case report suggests that English bull dogs may be more susceptible to canine herpesvirus problems even at later age potentially due to deficiencies in the immune system. Acquired immunity has major protective role in herpesviruses.

In humans, vertical transmission of herpesvirus has been reported (10). Caesarean section can reduce transmission of herpes to neonates (10). The English bull dog puppies were delivered by caesarean section in this study. Several of the pregnant females in this English bulldog kennel were found to shed CHV in the vaginal, oral and nasal secretions before whelping at high titers based on direct fluorescent antibody test. Thus, the kennel owner elected to not allow sucking of the puppies on the mothers. In newborn puppies, almost all the passive antibodies from the mothers are transferred through colostrum and not transplacentally (endotheliochorial type of placentation) (11).

Canine herpesvirus problems are quite common based on investigations in the US in the last 10 years. Out of the 434 specimens examined for CHV, 61 were found to be positive; 28 were found to be suspected positive; and rest were negative. This indicates that CHV is frequently involved in various cases of mortality in the US.

### CONCLUSIONS

This study was based in part on observations of canine herpesvirus (CHV-1) infections circulating in a large breeding kennel of English Bull dogs with clinical disease, death and lesions in older dogs (9 weeks of age) in South-Central USA. The CHV-1 virus had been repeatedly detected in the kennel in several animals for at least 3 years based on clinical signs and confirmation of CHV-1 infections using virus isolation and direct CHV fluorescent antibody tests, antibody detection based on CHV-serum neutralization, virus DNA detection using CHV specific-PCR targeted to glycoprotein B (gB) of CHV, and histopathology lesions compatible with CHV. Several forms of the CHV-1 related syndromes were observed in the kennel including acute fatality in young and older puppies, respiratory disease, abortions, fetal reabsorption, mummification, corneal ulcers in adult dogs over 5 years of age and conjunctivitis in young puppies. Using CHV gB specific PCR, it was possible to detect CHV DNA in archived paraffin-embedded, formalin-fixed tissue blocks.

Due to the expense in breeding the English bull dogs, the client elected plasma therapy that has been tried for human varicella zoster herpesvirus (VariZiG). According to the clinic records, about 250 ml of plasma was collected from adult females. The plasma was obtained by mixing whole blood with anticoagulant. After centrifugation at 3,500×g, the clear plasma was collected using sterility precautions. About 20 ml, subcutaneous, of the pooled immune plasma was administered to sick newborn puppies. Based on the mortality, improvement in survival and well-being of treated puppies was observed compared to untreated puppies.

Currently, there is no approved vaccine for CHV-1 in the USA. However, there is an approved vaccine for controlling reproductive consequences of CHV-1 in the European Union (CHV-1 F205 strain, Merial Animal Health) (12). The availability of CHV-1 vaccine in the US based on the novel isolates described herein will reduce the numbers of CHV-1 associated infections. In herpes zoster, attenuation of disease has been reported in all cases that received immune plasma (13). Anti-herpes zoster antiserum has been clinically efficacious for post-exposure treatment (14).

### Example 2

### Preparation and Testing of Canine Herpesvirus Vaccine and/or Immunogenic Compositions

Currently, there is no approved vaccine for CHV-1 in the USA. However, there is an approved vaccine for controlling reproductive consequences of CHV-1 in the European Union (CHV-1 F205 strain, Merial Animal Health) (12). Availability of CHV-1 vaccine as described herein using an American isolate of CHV-1 (12010974) will help reduce the numbers of CHV-1 associated infections in the US. In herpes zoster, attenuation of disease has been reported in all cases that received immune plasma (13). Anti-herpes zoster antiserum has been clinically efficacious for post-exposure treatment (14).

Modified Live virus vaccine will be prepared by passaging the CHV-1 isolate (12010974) about 50-80 passages to cause CHV-1 attenuation. About 10⁻⁴ virus particles will be used in each immunizing dose of the vaccine. The virus attenuates by accumulating random mutations in the DNA genome. The trial vaccine will be antigenic but not cause disease and able to induce antibody titers above >1:4 when measured by serum neutralization (SN) tests.

CHV-1 Neutralization Test: Sera from subject who have received the vaccine and from control subject who have not are diluted two-fold starting at 1:2 dilution. Twenty-five micro-liter of Minimum Essential Media (MEM) medium with serum (test samples) and without serum (controls) is added to wells of multi-well plates. In tubes, the virus is diluted to final working concentration of 50 TCID₅₀ (50% Tissue Culture Infectious Dose) and various dilutions are added to the MEM medium in the wells of the multi-well plates. Plates are incubated at 37° C. for 1 hour to allow the binding of the CHV-1 virus with serum antibodies. Thereafter, about 150 ul of a 100,000 cells per ml suspension of cells (e.g. Madin-Darby canine kidney (MDCK) cells) is added to appropriate test and control wells. Plates are incubated for 5 days at 37° C. The cell culture plates are then examined for cytopathology, such as rounding and detachment of cells. The SN titer is the reciprocal of the highest dilution of serum that inhibits 50% of the cell culture.

Challenge Protection Studies: Newborn puppies 1 day-1 month of age will be challenged with an infective dose of low passage of CHV-1, i.e. a dose that would be infective and would cause disease, if non-attenuated, wild type virus was used. Administration of the doses of attenuated virus is carried out by intramuscular or subcutaneous administration.

Efficacy of CHV-1 MLV Vaccine: Ten vaccinated and 10 unvaccinated controls will be compared for CHV-SN titers. A titer of 1:4 on CHV-1 SN will be taken as sufficient protection after vaccination. Similarly, in another study the vaccine efficacy on providing protection to puppies will be measured using shedding of CHV-1, lesion scores, Fluorescent antibody testing (FAT) antigen detection and PCR evaluation of CHV DNA.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

### REFERENCES

1. Ledbetter E C. 2013. Canine herpesvirus-1 ocular diseases of mature dogs. New Zealand veterinary Journal 61:193-201.

2. Schulz B S, Kurz S, Weber K, Balzer H J, Hartmann K. 2014. Detection of respiratory viruses and Bordetella bronchiseptica in dogs with acute respiratory tract infections. Vet J (In Press)

3. Erles K, Dubovi E J, Brooks H W, Brownlie J. 2004. Longitudinal study of viruses associated with canine infectious respiratory disease. J Clin Microbiol 42:4524-4529.

4. Reubel G, Perkin J, Webb-Wagg K, Hardy C. 2002. Nucleotide sequence of glycoprotein genes B, C, D, G, H, and I, the thymidine kinase and protein kinase genes and hene homologue UL24 of an Australian isolate of canine herpesvirus. Virus genes 25:195-200.

5. DeBiasi R L, Kleinschmidt-DeMasters B K, Weinberg A, Tyler K L. 2002. Use of PCR for the diagnosis of herpesvirus infections of the central nervous system. J Clin Viral 25:S5-S11.

6. Mills A M, Guo F P, Copland A P, Pai R K, Pinsky B A. 2013. A comparison of CMV detection in gastrointestinal mucosal biopsies using immunohistochemistry and PCR performed on formalin-fixed, paraffin-embedded tissue. Am J Surg Pathol 37:995-1000.

7. Kawakami K, Ogawa H, Maeda K, Imai A, Ohashi E, Matsunaga S, Tohya Y, Ohshima T, Mochizuki M. 2010. Nosocomial outbreaks of serious canine infectious tracheobronchitis (Kennel Cough) caused by canine herpesvirus infection. 48:1176-1181.

8. Malone E K, Ledbetter E C, Rassnick K M, Kim S G, Russell D. 2010. Disseminated canine herpesvirus-1 infection in an immunocompromised adult dog. J Vet Intern Med 2010. 24:965-968.

9. Carmichael L E, Barnes F D, Percy D H. 1969. Temperature as a factor in resistance of young puppies to canine herpesvirus. J Infect Dis 120:669-678.

10. Brown Z. 2004. Preventing herpes simplex virus transmission to the neonates. Herpes 11:175A-186A.

11. Chappuis G. 1998. Neonatal immunity and immunization in early age: lessons from veterinary medicine. Vaccine. 16:1468-1472.

12. Poulet H, Guigal P M, Soulier M, Leroy V, Fayet G, Minke J, Chappuis G. 2001. Protection of puppies against canine herpesvirus by vaccination of dams. Vet Rec 148:691-695.

13. Winsnes R. 1978. Efficacy of zoster immunoglobulin in prophylaxis of varicella in high-risk patients. Acta Paediatr Scand 67:77-82.

14. Koren G, Money D, Boucher M. 2002. Serum concentrations, efficacy, and safety of a new, intravenously administered varicella zoster immune globulin in pregnant women. J Clin Pharmacol 42:267-274.

What is claimed is:

1. A method of detecting canine herpes virus-1 (CHV-1) in a fixed tissue sample that is embedded in paraffin, comprising
i) removing paraffin from said fixed tissue sample that is embedded in paraffin;
ii) isolating DNA from said fixed tissue sample; and
iii) detecting a CHV-1-specific gene in DNA isolated from said fixed tissue sample.

2. The method of claim 1, wherein said fixed tissue sample is a formalin-fixed tissue sample.

3. The method of claim 1, wherein said CHV-1-specific gene encodes Glycoprotein B (gB).

4. The method of claim 1, wherein said step of detecting is performed by PCR.

5. The method of claim 1 further comprising the steps of, before said step of removing paraffin,
fixing said tissue sample in a fixative, and
embedding fixed tissue in paraffin.

6. A vaccine or immunogenic composition for eliciting an immune response against CHV-1 in a canine, comprising
i) an attenuated CHV-1 isolate deposited as ATCC Deposit No. ______, or progeny thereof, or a killed virus corresponding to CHV-1 isolate ______; and
ii) a physiologically compatible carrier.

7. A method for eliciting an immune response to CHV-1 in a subject in need thereof, comprising administering to said subject a composition comprising
i) an attenuated CHV-1 deposited as ATCC Deposit No. ______, or progeny thereof, or a killed virus corresponding to CHV-1 isolate ______; and
ii) a physiologically compatible carrier.

8. The method of claim 7, wherein said composition comprises said killed virus and said subject is a dog residing in a kennel that is known to have circulating CHV-1 and/or said subject is a pregnant dog.

9. The method of claim 7, wherein said composition comprises said attenuated CHV-1 and said subject is a dog residing in a kennel in which CHV-1 is enzootic, and/or said subject is a dog that is not pregnant.

10. The method of claim 7, wherein a single dose of said composition comprises from about four to about six logs of virus antigen.

11. A method of intervening to prevent the transmission of CHV-1 infection in breeding kennels, said method comprising two or more of the following steps;
i) prescreening dogs for CHV-1 before introduction into a breeding kennel;
ii) screening for CHV-1 viral shedding by dogs in a kennel by testing for the presence of CHV-1 in a pooled sample of: secretions from external orifices and/or from external epithelial surfaces of said dogs;
iii) periodically monitoring a pool of external epithelial surfaces of said dogs for the presence of CHV-1;
iv) disinfecting kennel surfaces with 0.01% bleach;
v) avoiding contact between dogs in the kennel and wildlife; and
vi) building individual and/or herd immunity by administering to said dogs in said kennel the vaccine or immunogeninc composition of claim 6.

12. The method of claim 11, wherein said step of periodically monitoring is performed yearly.

13. The method of claim 11, wherein said step of periodically monitoring is performed by PCR.

14. The method of claim 11, wherein a killed virus composition is administered to pregnant dogs in said kennel and an attenuated live virus CHV-1 composition is administered to non-pregnant dogs in said kennel.

15. A method of preventing vaginal shedding of CVH-1 in a female dog infected with CHV-1, comprising
contacting vaginal surfaces of said female dog with a composition comprising at least one anti-herpesvirus compound.

16. The method of claim 15, wherein said female dog is pregnant and said step of contacting prevents transmission of said CHV-1 to offspring during birth.

17. The method of claim 15, wherein said at least one anti-herpesvirus compound is selected from the group consisting of acyclovir, acyclovir elaidate, idoxuridine, cytarabine, vidarabine, trifluorothymidine, and 9-8-D-arabinofuranosyl-2,6-diaminopurine (ara-DAP).

18. A vaginal sleeve to prevent transmission of infectious agents from tissue of a cervix and/or a vagina of a mother to an offspring during birth, comprising
a substantially cylindrical tube with a distal end and a proximal end, said substantially cylindrical tube possessing an axis with length and a cross sectional area
wherein
a length of said axis of said substantially cylindrical tube is equal to or greater than a length of said vagina;
a cross-sectional area of said tube is sufficient to allow passage of said offspring through said sleeve;
said distal end comprises an opening bounded by an edge, said edge having a circumference and width sufficient to make direct contact with and cover cervical and/or vaginal tissue adjacent to an opening formed by dilation of said cervix, thereby surrounding said opening; and
said proximal end comprising an opening with a diameter sufficient to allow said offspring to pass through.

19. The vaginal sleeve of claim 18, further comprising at least one purse string-type closure mechanism.

20. The vaginal sleeve of claim 18, wherein at least a portion of said vaginal sleeve is made from transparent material.

21. The vaginal sleeve of claim 18, wherein said distal end comprises a telescoping ring.

22. A method of preventing transmission of CHV-1 from vaginal tissue of a pregnant female to offspring during birth, comprising
inserting the vaginal sleeve of claim 18 into a vagina of said pregnant female prior to birth of said offspring; and
delivering said offspring through said vaginal sleeve.

23. The method of claim 22, wherein said pregnant female is a canine.
